(19) United States
(12) Patent Application Publication
(10) Pub. No.: US 2025/0052771 A1
(43) Pub. Date: Feb. 13, 2025

(54) DIAGNOSTIC LIPID BIOMARKERS FOR COVID-19 AND METHODS FOR USE THEREOF

(71) Applicants: NATIONAL RESEARCH COUNCIL OF CANADA, Ottawa (CA); UNIVERSITY OF OTTAWA, Ottawa (CA)

(72) Inventors: Miroslava Cuperlovic-Culf, Ottawa (CA); Mohammad Sajjad Ghaemi, Toronto (CA); Irina Alecu, Ottawa (CA); Steffany Bennett, Ottawa (CA)

(21) Appl. No.: 18/720,354

(22) PCT Filed: Dec. 16, 2022
(86) PCT No.: PCT/CA2022/051843

§ 371 Date: Jun. 14, 2024

Related U.S. Application Data
(60) Provisional application No. 63/291,159, filed on Dec. 17, 2021.

(51) Int. Cl. G01N 33/92

(52) U.S. Cl. CPC G01N 33/92; G01N 2333/165; G01N 2570/00; G01N 2800/26

(57) ABSTRACT

The disclosure relates to methods for diagnosing, detecting or screening for COVID-19 in a subject. The method comprises measuring the concentration of at least two sphingolipids in a blood sample. The sphingolipids are selected from Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0). The method further comprises determining a ratio of the concentration of the at least two sphingolipids and comparing the ratio with a reference value, wherein a differential between the ratio and the reference value is indicative that the subject has or likely has COVID-19. Also provided is a kit for use with the method disclosed herein.

### RELATED APPLICATIONS

This is a Patent Cooperation Treaty Application which claims the benefit of priority of U.S. Provisional Patent Application No. 63/291,159, filed Dec. 17, 2021 which is incorporated herein by reference in its entirety.

### FIELD

This disclosure pertains to methods for diagnosing coronavirus disease 2019 (COVID-19) and more particularly to lipid biomarker panels for diagnosis of COVID-19.

### BACKGROUND

In spite of vaccination efforts, the COVID-19 pandemic remains a major world-wide challenge with current, severely skewed, distribution of vaccines across the world still fueling spread of the disease. Progression of new variants is further continuing to cause concern, possibly leading to more virulent strains and even leading to speculations regarding reduced activity of currently used vaccines (Gostin 2021, Darby and Hiscox 2021). The incubation period for severe acute respiratory syndrome virus 2 (SARS-CoV-2), the virus responsible for COVID-19, ranges from 2 to 14 days, leading to a high risk for uncontrollable spread particularly when combined with the fact that a fraction of infected individuals never develop symptoms. Additionally, during periods of this pandemic, world-wide reliance on one testing approach has caused supply chain issues in some locations further delaying test results and increasing regulation problems.

A number of diagnostic technologies have been developed and used for detecting SARS-CoV-2 infection (aka COVID-19) including molecular genetic assays detecting viral RNA from a clinical sample, isothermal nucleic acid amplification assays, hybridization microarray assays, serological and immunological assays for anti-SARS-CoV-2 antibodies and chest CT scan analysis with AI enhanced detection (Carter et al. 2020, Wang et al. 2020, Harmon et al. 2020). Nucleic acid-based tests use polymerase chain reaction (PCR) to quantify viral RNA based on specifically selected complementary probe sequences. Selection of an optimal sequence remains an issue with, for example, different agencies recommending either specific SARS-CoV-2 RNA regions (specifically viral nucleocapsid N1, N2 and human RNase P gene) or RNA-dependent RNA polymerase (RdRP) and envelope genes (Weissleder et al. 2020). Clinical sensitivity of PCR tests decreases over the course of the disease with over 90% clinical sensitivity during the first 5 days from symptom onset but falling to around 70% from days 9 to 11 and even 30% at day 21 (Miller et al. 2020). This has prompted suggestions to combine PCR analysis with enzyme-linked immunosorbent assays, measuring anti-SARS-CoV2 antibodies and in particular IgM, IgG and IgA, as this test reaches 100% accuracy post day 21. Logistics and cost of utilizing two different tests for each suspected case remain a main problem.

Small molecules, including both hydrophilic and lipophilic, metabolites and lipids, remain the most utilized biomarkers for the majority of conditions as they provide information about the disease as well as the host's response and indicate both metabolic and immune response. Metabolomic and lipidomic screens and possible biomarker panels offer the possibility for screening from plasma, exosomes (Song et al. 2020) or saliva (Sapkota et al. 2021).

Previous analysis of the metabolic markers for those with predispositions that could lead to severe COVID-19 (Julkunen et al. 2021) have shown significant changes in lipoprotein lipids, impaired fatty acid balance, and high chronic inflammation, as well as changes in levels of some amino acids. The strongest predictor for future risk of severe SARS-CoV-2 infection in this study was GlycA which was previously linked with increased neutrophil activity and the risk for severe and fatal infections. Furthermore, earlier work has shown possible association between blood lipid levels and N-glycosylation related to inflammation (Liu et al. 2018).

Metabolic markers for poor prognosis have been proposed, specifically an observed increase in anthranilic acid, derived from the kynurenine pathway (Danlos et al. 2021), as well as the ratio of arginine and kynurenine which provides 100% accuracy in classification from healthy controls (Fraser et al. 2020). Additionally, levels of several amino acids are changed in COVID-19 based on severity (Danlos et al. 2021), while also allowing for the prediction of severity in healthy subjects (Julkunen et al. 2021).

Lipids have been linked to inflammatory response (Theken and FitzGerald 2021) and N-glycosylation of immunoglobulin G-IgG (Liu et al. 2018), an important component of immunological response. Additionally, a recent publication has shown lipid differences in COVID-19 patients relative to healthy controls as well as differences in lipid profiles across a range of severity levels (Caterino et al. 2021).

Studies thus far have focused on determining overall differences between COVID-19 patients and healthy controls, often targeting COVID-19 patients at one time point only. With COVID-19 presenting a particular challenge for the elderly, there remains a need to determine smaller subsets of biomarkers that can distinguish patients with COVID-19 disease from patients hospitalized with other diseases, in particular dementias, as well as from healthy subjects. Determination of such a subset of molecular markers that can provide accurate diagnosis at different disease stages and that can be measured with different technologies is desirable.

Risk of neurological diseases as a consequence of SARS-CoV-2 infection has been indicated (Nolen et al. 2022) as well as a higher risk of COVID-19 complications in patients with neurological diseases. There is a lack of diagnostic molecular markers that can distinguish SARS-CoV-2 infection in both healthy patients and patients with Mild Cognitive Impairment (MCI), Parkison's disease (PD), Parkinson's disease with dementia (PDD), Dementia Lewy Bodies (DLB), or Alzheimer's disease (AD). There is also a lack of diagnostic markers for the risk of neurological disease in relation to COVID-19. Determination of a subset of markers relating to COVID-19 diagnosis with an objective diagnosis of neurological disease is needed.

### SUMMARY

The inventors have identified several lipid biomarkers that are differentially altered in COVID-19 patients. Specifically, the inventors have identified ratios of lipid concentrations of specific sphingolipid species that are decreased or increased in COVID-19 patients. In addition, the inventors have determined that these lipid concentration ratios can provide a highly accurate biomarker panel for diagnostic separation of COVID-19 patients from healthy subjects, as well as subjects with other diseases such as dementias.

In an aspect there is provided herein a method of diagnosing, detecting or screening for COVID-19 in a subject, the method comprising: a) obtaining a blood sample from the subject; b) measuring concentration of at least two sphingolipids in the blood sample, the sphingolipids being selected from Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0); c) determining a ratio of the concentration of at least the two sphingolipids; and d) comparing the ratio with a reference value, wherein a differential between the ratio and the reference value is indicative that the subject has or likely has COVID-19.

In an embodiment, the concentrations of Cer(d18:1/26:0) and Cer(d18:1/16:0) are determined.

In an embodiment, a [Cer(d18:1/26:0)]/[Cer(d18:1/16:0)] ratio is determined and when the ratio [Cer(d18:1/26:0)]/[Cer(d18:1/16:0)] is less than a reference value of 3, the subject is identified as having or likely having COVID-19.

In an embodiment, the concentration of HexCer(d18:1/22:0) and 1-O-nervonoyl-Cer(d18:1/18:2) is determined.

In an embodiment, a [HexCer(d18:1/22:0)]/[1-O-nervonoyl-Cer(d18:1/18:2)] ratio is determined and when the [HexCer(d18:1/22:0)]/[1-O-nervonoyl-Cer(d18:1/18:2)] ratio is less than a reference value of 10 the subject is identified as having or likely having COVID-19.

In an embodiment, the concentrations of Cer(d18:1/16:0), Cer(d18:1/26:0), 1-O-nervonoyl-Cer(d18:1/18:2) and HexCer(d18:1/22:0) are determined.

In an embodiment, a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is determined and when the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is less than a reference value of 14 the subject is identified as having or likely having COVID-19.

In a further embodiment, the concentrations of Cer(d18:1/26:0), HexCer(d18:1/22:0), Cer(d18:1/16:0), and Cer(d18:1/18:0) are determined.

In an embodiment, a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is determined and when the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is less than a reference value of 3.2 the subject is identified as having or likely having COVID-19.

In an embodiment, the blood sample is a whole blood sample, a blood serum sample, a dried blood spot, or a blood plasma sample, optionally a blood plasma sample.

In an embodiment, the sphingolipid concentrations are measured using mass spectrometry, nuclear magnetic resonance spectroscopy, fluorescence spectroscopy, dual polarization interferometry, a high performance separation method, an immunoassay and/or with a binding moiety capable of specifically binding the sphingolipid analyte, optionally the sphingolipid concentrations are measured using mass spectrometry.

In an embodiment, the reference value is derived from a healthy subject or a population of healthy subjects, optionally the subject(s) is/are matched for age, sex and/or underlying disease(s).

In an embodiment, the subject has an underlying disease, optionally dementia and/or Parkinson's disease. In an embodiment, the underlying disease is a neurological disease. In a further embodiment, the neurological disease is Mild Cognitive Impairment (MCI), Parkison's disease (PD), Parkinson's disease with dementia (PDD), Dementia Lewy Bodies (DLB), or Alzheimer's disease (AD).

In an embodiment, the method further comprises administering a treatment to the subject identified as having or likely having COVID-19. The treatment can be, for example, a treatment for COVID-19 or for one or more complications of COVID-19.

Also disclosed herein in another aspect is a kit comprising internal standards for mass spectrometry analysis for quantification of two or more sphingolipids selected from Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0), and optionally comprising calibration standards, quality controls, test samples for assay validation and/or instructions for use thereof.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the disclosure are described with reference to the drawings:

FIGS. 1A and 1B are two-dimensional plots following stochastic neighbour embedding (T-SNE) (FIG. 1A) and Principal Component analysis (PCA) (FIG. 1B). Dots represent individual samples, where filled circles indicate COVID-19 positive subjects with other points representing non-COVID-19 controls including samples collected prior to the COVID-19 pandemic from healthy controls (preCOVIDCTRL), dementia patients (preCOVIDNeuroCTRL), pre-COVID hospitalized patients (PreCOVIDHospCTRL), as well as COVID-19 negative subject samples collected during the pandemic (CTRLCOVID).

FIG. 2 is a schematic of the methodology used for determination of the optimal lipid marker subset. Ensemble feature selection is done with several highly distinct approaches providing a subset of features of significance regardless of the method. Clustering and correlation methods are used to narrow down the selected feature set into non-correlated features.

FIGS. 3A, 3B, 3C, 3D, and 3E show a series of graphs showing the selection of the most significant features using: F-statistics (FIG. 3A), Relieff (FIG. 3B), Lasso (FIG. 3C), Bagging of Trees (FIG. 3D), and Random Forest (FIG. 3E).

FIG. 4A and FIG. 4B show Fuzzy C-means (FCM) clustering of features across all groups where FIG. 4A shows clustering of groups based on the most significant cluster membership as well as Principal Component Analysis of samples based on fuzzy clustering memberships for 10 clusters and FIG. 4B. shows membership values for each feature when grouped between 20 clusters; FIG. 4C depicts a Principal component analysis of FCM membership values. Indicated with asterisks are features selected as significant by all methods used and showing distinct cluster membership, namely 1-O-nervonoyl-Cer(d18:1/18:2), Cer(d18:1/18:0), HexCer(d18:1/22:0), Cer(d18:1/26:0), Cer(d18:1/16:0) and PE-Cer(d18:1/24:0). FCM is performed for 10 clusters with m=2. Shown are membership values over 0.1.

FIG. 5 shows a series of signed distance correlation networks of selected major features with correlation values above 0.6 and p<0.01.

FIGS. 6A, 6B, 6C, 6D, 6E, 6F, 6G and 6H show a series of boxplots showing concentration levels of lipids HexCer(d18:1/22:0) (FIG. 6A), 1-O-nervonoyl-Cer(d18:1/18:2) (FIG. 6B), Cer(d18:1/26:0) (FIG. 6C) and Cer(d18:1/16:0) (FIG. 6D) and concentration ratios Cer(d18:1/26:0)/Cer(d18:1/26:0) (FIG. 6E), HexCer(d18:1/22:0)/1-O-nervonoyl-Cer(d18:1/18:2) (FIG. 6F) and [Cer(d18:1/26:0)*HexCer(d18:1/22:0)]/[Cer(d18:1/16:0)*1-O-nervonoyl-Cer(d18:1/18:2)] (FIG. 6G), Cer(d18:1/26:0)*HexCer(d18:1/22:0)]/[Cer(d18:1/16:0)*Cer(d18:1/18:0)] (FIG. 6H) in COVID-19 patients and control cohorts i.e. healthy (PreCOVIDCTRL), dementia (PreCOVIDNeuroCTRL) and in COVID-19 negative (CTRLCOVID) subjects. Based on two-sample Kolmogorov-Smirnov test of difference between values for COVID and non-COVID groups p=2.95*10-25-highly significant.

FIGS. 7A and 7B show concentration ratios of Cer(d18:1/26:0)*HexCer(d18:1/22:0)]/[Cer(d18:1/16:0)*1-O-nervonoyl-Cer(d18:1/18:2)] (FIG. 7A) and Cer(d18:1/26:0)*HexCer(d18:1/22:0)]/[Cer(d18:1/16:0)*Cer(d18:1/18:0)] (FIG. 7B) in COVID-19 patients and healthy control cohorts, as well as in patients with neurological disorders including AD, DLB, PD, PDD and Vascular Dementia with significantly different level in COVID-19.

FIG. 8 shows example chemical structures of the sphingolipids that can be measured with the method disclosed herein.

### DETAILED DESCRIPTION OF THE DISCLOSURE

### I. Definitions

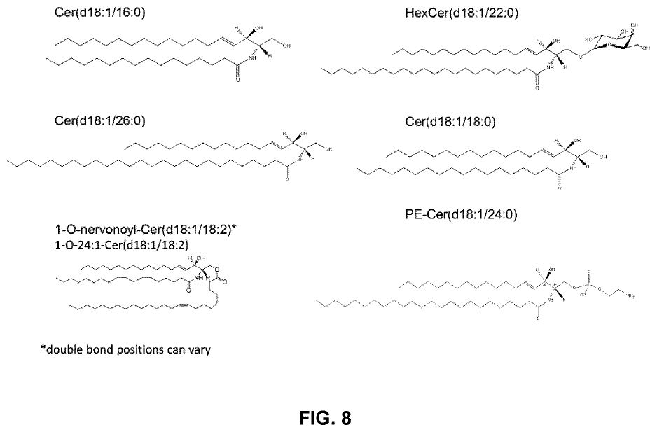

The term “sphingolipid” as used herein means a member of a class of lipids containing the organic aliphatic amino alcohol sphingosine or a substance structurally similar to it e.g. sphinganine. Sphingolipids include for example ceramides such as Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0). As referred to herein, sphingolipids are named in accordance with the following nomenclature: “CE” is cholesteryl ester, “Cer” is ceramide, “DAG” is diacylglycerol, “HexCer” is hexosyl-ceramide, “Gb3” is globotriaosylceramide, “Gb4” is globotetraosylceramide, “LacCer” is lactosylceramides, “PE” is phosphatidylethanolamine, “SM” is sphingomyelin and “Sph” is sphingosine. In accordance with the nomenclature “dX:Y/A:B”, the d indicates that the sphingoid base backbone of the molecule has two OH groups, X indicates the number of total carbon atoms and Y indicates the total number of double bonds in the sphingosine backbone, while A indicates the number of total carbon atoms and B indicates the total number of double bonds in the fatty acid portion of the molecule. Examples of sphingolipids that are measured using the methods disclosed herein include the lipids shown in FIG. 8 as well as enantiomers, salts, and adducts thereof. Adducts include for example [M+H]⁺, [M+H—H₂O]⁺, [M.]⁺, [M+2H]²⁺, [M+3H]³⁺, [M+4H]⁴⁺, [M+K]⁺, [M+2K]²⁺, [M+2K—H]⁺, [M+Na]⁺, [M+2Na]²⁺, [M+2Na—H]⁺, [M+Li]⁺, [M+2Li]²⁺, [M+NH4]⁺, [M−H]⁻, [M−2H]²⁻, [M−3H]³⁻, [M−4H]⁴⁻, [M+Cl]⁻, [M+OAc]⁻, [M+HCOO]⁻ and M (neutral).

The term “biomarker” as used herein refers to any molecule that can be used as an indicator of a biological state, in the diagnosis/prognosis of a disease or disorder, and/or in the prediction of the outcome of a treatment or procedure. A biomarker may be used on its own, or in combination with other biomarkers and/or methods. A biomarker can be for example a lipid.

The term “COVID-19” as used herein refers to coronavirus disease 2019, a disease caused by the SARS-CoV-2 virus. Symptoms of COVID include respiratory tract infections such as lower respiratory tract infections, high fever, dry cough, shortness of breath, pneumonia, gastro-intestinal symptoms such as diarrhea, organ failure (kidney failure and renal dysfunction), septic shock, and death in severe cases.

The term “SARS-CoV-2” as used herein refers to the newly-emerged severe acute respiratory syndrome virus 2 (SARS-CoV-2), and variants thereof, which was identified as the cause of a serious outbreak starting in Wuhan, China, and which has rapidly spread to other areas of the globe. The term “variant” as used herein refers to any one or more mutations in the coronavirus, whether naturally occurring or engineered. Non limiting examples of SARS-CoV-2 variants include alpha (B.1.1.7), beta (B.1.351, B.1.351.1,B.1.351.2, B.1.351.3, B.1.351.4), delta (B.1.617.2, AY.1, AY.2, AY.3, AY.3.1), gamma (P.1, P.1.1, P.1.2), and omicron (B.1.1.529) variants.

The term “determining” as used herein includes for example measuring a level such as a concentration and/or obtaining measured data.

The term “blood sample” as used herein includes for example whole blood, blood serum, blood plasma, or dried blood spot. The blood sample can be collected from the subject using techniques well known to a person skilled in the art.

The term “reference value” as used herein means a cut-off level above or below which the subject is identified as having or likely having COVID-19. The reference value can be previously determined or calculated or be yet to be determined or calculated. For example, a blood sample may be obtained from a non-COVID-19 control subject and analyzed in respect to the lipids of interest. Lipid concentration ratios may then be determined to provide the reference value.

The term “differential” as used herein means a difference between a ratio of the concentration of at least two sphingolipids in a blood sample of a subject and a reference value that is indicative of COVID-19. The differential can be a positive value or a negative value, meaning the ratio from the subject is higher or lower than the reference value respectively.

The term “subject” as used herein includes all members of the animal kingdom including mammals, and suitably refers to humans.

The term “level” as used herein refers to an amount (e.g. concentration as well as parameter values calculable based thereon such as a ratio) of a biomarker (i.e. lipid related level) that is detectable, measurable or quantifiable in a test biological sample and/or a reference biological sample, for example, a blood sample. For example, the level can be a concentration such as pmol/mL, μg/L, ng/ml or pg/mL, a relative amount or ratio such as 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10, 15, 20, 25, and/or 30 times more or less than a reference value of a control biomarker. The reference value can, for example, be the average or median level of a biomarker in a plurality of blood samples.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least 5% of the modified term if this deviation would not negate the meaning of the word it modifies.

More specifically, the term “about” means plus or minus 0.1% to 50%, 5% to 50%, 10% to 40%, 10% to 20%, or 10% to 15%, preferably 5% to 10%, most preferably about 5% of the number to which reference is being made.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be understood by a person skilled in the art. For example, in the following passages, different aspects of the disclosure, are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Methods

Disclosed herein are lipid biomarkers that can be used to assess whether a subject has or likely has COVID-19.

The inventors have identified lipid biomarkers that are differentially expressed in COVID-19 subjects compared to subjects without COVID-19. Specifically, using an ensemble of feature selection methods with feature characterization through clustering and distance correlation analysis, blood lipid biomarkers have been identified as being increased or decreased in COVID-19 subjects compared to non-COVID-19 subjects. Also disclosed herein are lipid biomarkers that, when used as specific ratios of concentration measurements, provide accurate diagnosis of COVID-19 disease at any stage and severity level relative to both healthy subjects and patients with dementias. Advantageously, the biomarker ratios do not require internal standards.

In accordance with an aspect of the present disclosure there is provided a method of diagnosing, detecting or screening for COVID-19 in a subject. The method comprises obtaining a blood sample that has been obtained from the subject and measuring concentration of at least two sphingolipids in a subject blood sample, the at least two sphingolipids being selected from Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0). A ratio of the concentration of the at least two sphingolipids is determined. The ratio is compared with a reference value, wherein a differential between the ratio and the reference value is indicative the subject has or likely has COVID-19.

Measuring concentration of at least two sphingolipids in the blood sample means for example measuring a concentration of a first sphingolipid and measuring a concentration of second sphingolipid. For example, if there are more than two sphingolipids, a concentration of each is measured. Also, a skilled person would understand that depending on which concentration(s) is/are in the denominator and which concentration(s) is/are in the numerator of the ratio, the differential indicative of COVID-19 can be a positive value or a negative value.

In an embodiment, the concentration of two sphingolipids in the blood sample is determined. In an embodiment, the concentration of three sphingolipids in the blood sample is determined. In an embodiment, the concentration of four sphingolipids in the blood sample is determined. In an embodiment, the concentration of five sphingolipids in the blood sample is determined. In an embodiment, the concentration of six sphingolipids in the blood sample is determined.

In an embodiment, the concentrations of Cer(d18:1/26:0)] and [Cer(d18:1/16:0)] are determined.

In an embodiment, a [Cer(d18:1/26:0)]/[Cer(d18:1/16:0)] ratio is determined and when the [Cer(d18:1/26:0)]/[Cer(d18:1/16:0)] ratio is less than a reference value of 3 the subject is identified as having or likely having COVID-19.

In another embodiment, the concentrations of HexCer(d18:1/22:0) and 1-O-nervonoyl-Cer(d18:1/18:2) are determined.

In an embodiment, a [HexCer(d18:1/22:0)]/[1-O-nervonoyl-Cer(d18:1/18:2)] ratio is determined and when the [HexCer(d18:1/22:0)]/[1-O-nervonoyl-Cer(d18:1/18:2)] ratio is less than a reference value of 10 the subject is identified as having or likely having COVID-19.

In an embodiment, a [Cer(d18:1/26:0)]/[Cer(d18:1/16:0)] ratio and a HexCer(d18:1/22:0)]/[1-O-nervonoyl-Cer(d18:1/18:2)] ratio are determined and when the [Cer(d18:1/26:0)]/[Cer(d18:1/16:0)] ratio is less than a reference value of 3 and the [HexCer(d18:1/22:0)]/[1-O-nervonoyl-Cer(d18:1/18:2)] ratio is less than a reference value of 10 the subject is identified as having or likely having COVID-19.

In a further embodiment, the concentrations of Cer(d18:1/16:0), Cer(d18:1/26:0), 1-O-nervonoyl-Cer(d18:1/18:2) and HexCer(d18:1/22:0) are determined.

In an embodiment, a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is determined and when the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is less than a reference value of 14, the subject is identified as having or likely having COVID-19.

In an embodiment, a [Cer(d18:1/26:0)]/[Cer(d18:1/16:0)] ratio and a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio are determined and when the [Cer(d18:1/26:0)]/[Cer(d18:1/16:0)] ratio is less than a reference value of 3 and the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is less than a reference value of 3 and the is identified as having or likely having COVID-19.

In an embodiment, a [HexCer(d18:1/22:0)]/[1-O-nervonoyl-Cer(d18:1/18:2)] ratio and a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio are determined and when the [HexCer(d18:1/22:0)]/[1-O-nervonoyl-Cer(d18:1/18:2)] ratio is less than a reference value of 10 and the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is less than a reference value of 14, the subject is identified as having or likely having COVID-19.

In an embodiment, a [Cer(d18:1/26:0)]/[Cer(d18:1/16:0)] ratio, [HexCer(d18:1/22:0)]/[1-O-nervonoyl-Cer(d18:1/18:2)] ratio and a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio are determined and when the [Cer(d18:1/26:0)]/[Cer(d18:1/16:0)] ratio is less than a reference value of 3, the [HexCer(d18:1/22:0)]/[1-O-nervonoyl-Cer(d18:1/18:2)] ratio is less than a reference value of 10 and the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is less than a reference value of 14, the subject is identified as having or likely having COVID-19.

In a further embodiment, the concentrations of Cer(d18:1/26:0), HexCer(d18:1/22:0), Cer(d18:1/16:0), and Cer(d18:1/18:0) are determined.

In an embodiment, a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is determined and when the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is less than a reference value of 3.2, the subject is identified as having or likely having COVID-19.

In an embodiment, a [Cer(d18:1/26:0)]/[Cer(d18:1/16:0)] ratio and a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio are determined and when the [Cer(d18:1/26:0)]/[Cer(d18:1/16:0)] ratio is less than a reference value of 3 and the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is less than a reference value of 3.2, the subject is identified as having or likely having COVID-19.

In an embodiment, a [HexCer(d18:1/22:0)]/[1-O-nervonoyl-Cer(d18:1/18:2)] ratio and a a,

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio are determined and when the [HexCer(d18:1/22:0)]/[1-O-nervonoyl-Cer(d18:1/18:2)] ratio is less than a reference value of 10 and the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

as having or likely having COVID-19.

In an embodiment, a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio and a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio are determined and when the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is less than a reference value of 14 and the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is less than a reference value of 3.2, the subject is identified as having or likely having COVID-19.

In an embodiment, a [Cer(d18:1/26:0)]/[Cer(d18:1/16:0)] ratio, a HexCer(d18:1/22:0)]/[1-O-nervonoyl-Cer(d18:1/18:2)] ratio and a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio are determined and when the [Cer(d18:1/26:0)]/[Cer(d18:1/16:0)] ratio is less than a reference value of 3, the [HexCer(d18:1/22:0)]/[1-O-nervonoyl-Cer(d18:1/18:2)] ratio is less than a reference value of 10 and the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is less than a reference value of 3.2, the subject is identified as having or likely having COVID-19.

In an embodiment, a [Cer(d18:1/26:0)]/[Cer(d18:1/16:0)] ratio, a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio and a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio are determined and when the [Cer(d18:1/26:0)]/[Cer(d18:1/16:0)] ratio is less than a reference value of 3, the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is less than a reference value of 14 and the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is less than a reference value of 3.2, the subject is identified as having or likely having COVID-19.

In an embodiment, a [HexCer(d18:1/22:0)]/[1-O-nervonoyl-Cer(d18:1/18:2)] ratio, a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio and a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio are determined and when the [HexCer(d18:1/22:0)]/[1-O-nervonoyl-Cer(d18:1/18:2)] ratio is less than a reference value of 10, the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is less than a reference value of 14, and the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is less than a reference value of 3.2, the subject is identified as having or likely having COVID-19.

In an embodiment, a [Cer(d18:1/26:0)]/[Cer(d18:1/16:0)] ratio, a [HexCer(d18:1/22:0)]/[1-O-nervonoyl-Cer(d18:1/18:2)] ratio, a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio and a

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio are determined and when the [Cer(d18:1/26:0)]/[Cer(d18:1/16:0)] ratio is less than a reference value of 3, the [HexCer(d18:1/22:0)]/[1-O-nervonoyl-Cer(d18:1/18:2)] ratio is less than a reference value of 10, the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is less than a reference value of 14, and the

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is less than a reference value of 3.2, the subject is identified as having or likely having COVID-19.

In an embodiment, the blood sample is a whole blood sample, a blood serum sample or a blood plasma sample.

In an embodiment, the blood sample is a blood plasma sample.

It will be understood that techniques well known to a person skilled in the art may be used for measuring sphingolipid concentrations. In an embodiment, the concentrations are measured using mass spectrometry, nuclear magnetic resonance spectroscopy, fluorescence spectroscopy, dual polarisation interferometry, a high performance separation method, an immunoassay and/or with a binding moiety capable of specifically binding the sphingolipid analyte. In an embodiment, the sphingolipid concentrations are measured using mass spectrometry.

In an embodiment, the reference value is derived from a healthy subject. In an embodiment, the reference value is obtained from a population of healthy subjects. Preferably, the subject(s) is/are matched for age, sex and/or underlying disease.

In an embodiment, the subject has no underlying disease. In other embodiments, the subject has an underlying disease, for example dementia and/or Parkinson's disease. In an embodiment, the underlying disease is a neurological disease. In a further embodiment, the neurological disease is Mild Cognitive Impairment (MCI), Parkison's disease (PD), Parkinson's disease with dementia (PDD), Dementia Lewy Bodies (DLB), or Alzheimer's disease (AD).

In an embodiment, the method further comprises administering a treatment to the subject identified as having or likely having COVID-19.

Also disclosed herein are kits for the detection of biomarker levels, i.e. sphingolipid levels that are used to assess whether a sample is from a subject having or likely having COVID-19.

For example, the kit can comprise extraction reagents for extracting sphingolipids from blood samples. The kit can comprise reagents, such as lipid standards, for analysis and/or quantification by a suitable method, such as mass spectrometry. The kit can further comprise instructions for use, including for example, instructions for identifying and/or quantifying the sphingolipids Cer(d18:1/16:0), Cer(d18:1/26:0), 1-O-nervonoyl-Cer(d18:1/18:2), HexCer(d18:1/22:0), Cer(d18:1/18:0) and/or PE-Cer(d18:1/24:0).

In an embodiment, the kit is for mass spectrometry analysis, comprising internal standards for quantification of two or more sphingolipids selected from Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0). The internal standards can be, for example, isotopically labelled. The kit can further comprise other reagents such as calibration standards, quality controls and/or test samples for assay validation.

Accordingly in an aspect there is provided a kit comprising a biomarker panel comprising two or more biomarker reagents, each biomarker reagent being specific for a corresponding biomarker selected from Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0), and optionally comprising a sample dilution buffer, a wash buffer, a control value and/or instructions for use thereof.

In an embodiment, the kit is for use in a method or product described herein.

Computer implemented methods and methods comprising machine learning applications are also described herein.

Another aspect of the disclosure includes a computer-readable medium comprising computer executable instructions, optionally for diagnostic software application recorded thereon, configured to be executed by one or more processors to cause a computer to perform a method of diagnosing, detecting or screening for COVID-19 in a subject, the method comprising: a) receiving concentration levels of at least two sphingolipids obtained from a blood sample of the subject, the sphingolipids being selected from Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0); b) determining a ratio of the concentrations of the at least two sphingolipids, c) comparing the ratio with a reference value, and d) predicting the subject as having or likely having COVID-19 when a differential between the ratio and the reference value is detected.

For example, the computer-readable medium can perform one or more of: a) automated reading from mass spec instrument output of measurement including peak areas and retention time for samples and quality control samples provided in a kit described herein; b) adjust measurements using standards (calibration standards, quality controls) provided in the kit; c) assign peaks of at least two sphingolipids obtained from a blood sample of the subject, wherein the at least two sphingolipids are selected from the following sphingolipids: Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0) and six isotopically labelled internal standards provided in the kit d) determine measurement areas for Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0) and six isotopically labelled internal standards provided in the kit. Alternatively, user directly inputs concentration levels of at least two sphingolipids obtained from a blood sample of the subject, wherein the at least two sphingolipids are selected from the following sphingolipids: Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0); e) software determines ratios of the concentration of the at least two sphingolipids as provided including one or more calculated as [Cer(d18:1/26:0)]/[Cer(d18:1/16:0)], [HexCer(d18:1/22:0)]/[1-O-nervonoyl-Cer(d18:1/18:2)],

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

and/or

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]} ,$$

f) software compares calculated ratios with a diagnostic reference value where if [Cer(d18:1/26:0)]/[Cer(d18:1/16:0)] is less than a reference value of 3, the subject is identified as having or likely having COVID-19; if [HexCer(d18:1/22:0)]/[1-O-nervonoyl-Cer(d18:1/18:2)] ratio is less than a reference value of 10 the subject is identified as having or likely having COVID-19; if

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

ratio is less than a reference value of 14 the subject is identified as having or likely having COVID-19 if

$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

ratio is less than a reference value of 3.2 the subject is identified as having or likely having COVID-19 and g) software provides user with diagnosis of individual ratios as well as combined score and diagnosis assessment using available ratios individually or in combination predicting the subject as having or likely having COVID-19 when a differential between the ratio and the reference value is detected. Combined score is provided from 2 or more ratios using union of binary ratio results weighted by individual ratio's accuracy measure. EM, SEM, McNemar's test or other algorithms provide combined binary diagnostic tests accuracy measure. User is presented with combined diagnostic information (COVID-19 likely or not) and accuracy or sensitivity or specificity or confidence interval or Mathew's index for diagnosis. Accuracy measures for combined diagnostic score are initially included in the software from available data and are updated regularly and automatically with new patient information provided by user.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Materials and Methods

90 lipids were measured in plasma samples of COVID-19 patients as well as non-COVID-19 controls, both healthy and hospitalized with dementias, to determine markers of SARS-CoV-2 infection at any disease stage and severity level.

Sphingolipids were extracted on ice using a modified Bligh and Dyer method, as previously described (Xu et al. 2013). Briefly, 100 μL of plasma were transferred to glass Kimble vials and inactivated with 300 uL of 100% ethanol (Commercial Alcohols, P016EAAN). Next, 3.7 mL of methanol (Fisher, catalog no. BP1105-4) acidified with 2% acetic acid (Fisher, catalog no. A38-212) were added to each sample. Lipid standards were added to each sample at time of extraction in the following amounts: 235 pmol of Cer(d18:1/16:0-D31) #868516, 470 pmol of GlcCer(d18:1/8:0) #860540, 470 pmol of GalCer(d18:1/8:0) #860538, and 101.6 pmol of SM (d18:1/18:1-D9) #791649 (Avanti Polar Lipids, Alabaster, USA), followed by 0.1M sodium acetate and chloroform (Fisher, catalog no. C298-500) and (J. T. Baker, catalog no. 9831-03) to a final ratio with acidified methanol of 2:1.7:2.1, respectively. Samples were vortexed after each step and then centrifuged for 5 min at 4° C. and 600×g. The organic phase was collected into a new tube, and the aqueous phase was back-extracted an additional 3 times using chloroform, with each subsequent organic phase collected being pooled with the previous one. The samples were dried under a constant stream of nitrogen, and lipids were re-solubilized in 300 μL of 100% ethanol (Commercial Alcohols, P016EAAN), flushed with nitrogen, and stored at −80° C.

The sphingolipids were analyzed by liquid chromatography-mass spectrometry on an Agilent 1290 Infinity II liquid chromatography system coupled to a QTRAP 5500 triple quadrupole-linear ion trap mass spectrometer with Turbo V ion source (AB SCIEX, Concord, Canada). Reverse phase chromatography was performed using a binary solvent gradient with solvent A (water with 0.1% formic acid (Fluka, catalog no. 56302) and 10 mM ammonium acetate (OmniPur, catalog no. 2145)) and solvent B (acetonitrile (J. T. Baker, Bridgewater, USA catalog no. 9829-03) and isopropanol (Fisher, catalog no. A461-4) at a ratio of 5:2 v/v with 10 mM ammonium acetate and 0.1% formic acid) pumped over a 100 mm×250-μm (inner diameter) capillary column packed with ReproSil®-Pur 120 C8 at a flow rate of 10 μl/min. The duration of the method was 50 minutes, with the gradient starting at 30% B and reaching 100% B over the first 5 minutes. This solvent composition was maintained until 35 minutes, and then ramped down to 30% B by 36 minutes and maintained until the end of the run. Data were acquired in positive ion mode using selected reaction monitoring (SRM), monitoring transitions from protonated molecular ions with Q3 ions of m/z 264.3 (double dehydration of d18:1 backbone), 282.3 (single dehydration of d18:1 backbone), 266.3 (double dehydration of d18:0 backbone), 262.3 (double dehydration of d18:2 backbone), 250.3 (double dehydration of d17:1 backbone), 252.3 (double dehydration of d17:1 backbone), or 184.1 (phosphocholine head group).

The molecular identities of lipid species were confirmed through an information-dependent acquisition (IDA)—enhanced product ion (EPI) experiment, where EPI spectra were analyzed for structural determination. Data acquisition was performed using Analyst software version 1.6.2 (AB SCIEX) and quantification analysis of SRM data was done in MultiQuant® 3.0.2 software version 3.0.8664.0 (SCIEX). Raw peak areas were normalized to the appropriate internal standards in order to account for extraction efficiency and instrument response, as well as to the volume of plasma extracted.

### Data Mining

Feature selection was performed using several different methods including statistical method based on F-test, and different machine learning methods including filter-method Relieff, lasso regularization method that selects out features (i.e. lipids) that do not contribute to the model, bagging of trees and random forest methods that combine boosting with decision tree algorithms for selection of the most significant features. Methods were selected to include requirements for Ensemble analysis approach to feature selection.

Univariate feature ranking using F-test was used to determine weight, significance of each feature for hypothesis that the response values are drawn from the same population and have the same mean as the predictor variables. Test determines p-value for statistical significance and results show score as: −log (p) as the measure of features importance.

Relieff is a K-nearest neighbors-based feature selection method able to determine conditional dependencies between features and present a feature estimation in regression and classification (Tibshirani 1996; I. Kononenko and Simec 1995; Igor Kononenko and Kukar 2007). Principal goal of Relieff is to estimate the weight or quality of features as classifiers based on how well their values can separate instances that are near to each other. For a randomly chosen feature Relieff finds k of its nearest neighbors from the same class and k nearest neighbors from different classes and iteratively optimizes selection based on their values for the chosen instance and its nearest neighbors. Specifically the weight for feature in a binary classification problem is calculated as: Wⱼⁱ=Wⱼⁱ⁻¹−Δⱼ(cᵣ,c_{q})/m·d_{rq} where Wⱼⁱ is the weight of feature j at the ith analysis iteration, Δⱼ(cᵣ, c_{q}) is 0 if observation cᵣ and c_{q} lead to different jth predictor and 1 either wise; m is the number of iterations and d_{rq} is the distance function between two observations. Analysis was performed using the relieff function in MATLAB® with k=10 and testing feature selection for COVID-19 patients samples including analysis for measurements at the first collection time point only and at all time points providing highly comparable results. Shown is the result for all COVID-19 samples. Samples were z-score normalized prior to analysis.

Lasso regression (Least Absolute Shrinkage and Selection Operator) (Tibshirani 1996) is a linear model with a cost function: 1/2N Σᵢ₌₁^{N}(yᵣₑₐₗ⁽ⁱ⁾−y_{pred}⁽ⁱ⁾)+∝Σⱼ₌₁ⁿ|aⱼ| where N is the number of observations, yᵣₑₐₗ⁽ⁱ⁾ and y_{pred}⁽ⁱ⁾ are the measured or predicted response at observation i, and the final sum, termed 11 penalty, includes an α hyperparameter that tunes the intensity of the penalty term. By minimizing cost function the Lasso algorithm automatically selects useful features and excludes features that are not useful in classification. Lasso analysis was performed using lasso function in MATLAB using 7-fold cross validation with α=0.2 Lasso feature selection was performed for COVID-19 patients samples only at the first collection time point. Lasso feature selection for all COVID-19 samples results in a smaller feature set that includes all features selected by Relieff (data not shown). Samples were z-score normalized prior to analysis.

Bagging of trees method uses ranking of features based on their relevance in the tree construction. In the bagging approach, feature selection is done on each bootstrap and results are combined. With bagging, the danger of overfitting is reduced and the result is more general. Each run uses a randomly selected sample subset. The number of leaves and trees for the analysis was optimized and finally selected to 5 leaves and 300 trees with the function TreeBagger running under MATLAB. Analysis was performed using all COVID-19 patient measurements in order to increase sample set size. Samples were z-score normalized prior to analysis.

Random Forest feature selection is an embedded method that combines functionalities of filter and wrapper methodologies. Similarly to the Bagging of trees the random forest method once again creates hundreds of random extractions of observations from the dataset and builds a decision trees for each group. However, unlike the bagging of trees approach, in random forest method not every decision tree sees all the features during tree building and in this way trees are de-correlated and less prone to over-fitting. The features' importance is determined for each decision tree node based on the purity of the branches. Random forest analysis was performed in Python® using in house routines and for COVID-19 patient measurements at the first time point.

Feature characteristics were assessed using clustering and correlation analysis using fuzzy c-means clustering used to select features with minimal co-behaviour and signed distance correlation analysis to determine functionally changing features.

Fuzzy C-means (FCM) clustering network. FCM clustering (developed by (Caterino et al. 2021; Dunn 1973), (Bezdek 1981)); introduced to omics (Bezdek 1981; Belacel et al. 2004) allows each feature to belong to more than one group by providing a degree of membership, “belonging”, to each cluster by maximizing proximity between similar features and distance between dissimilar features. FCM is based on the minimization of the objective function:: Jₘ=Σᵢ₌₁^{N}Σⱼ₌₁^{C}uᵢⱼ^{m}∥xᵢ−cⱼ∥² where (1,∞) is the “fuzzyfiction” factor, uᵢⱼ^{m} is the membership degree for feature xᵢ to the cluster j with cⱼ defining the cluster center. FCM clustering assigns objects to groups with features belonging to the same clusters showing more similarity to each other than to features in other clusters. Higher membership value indicates stronger belonging to the cluster with membership value of 1 ultimately indicating that feature is only associated with the single cluster. Fuzzy c-means clustering was used to select features with minimal co-behaviour.

Distance correlation network: Correlation was calculated on z-score normalized features using Pearson, Spearman and Distance correlation analysis where only distance correlation and signed distance correlation results were shown. MATLAB routine corr was used for Pearson and Spearman correlation. Distance correlation was calculated following the definition by Székely et al (2007) as follows:

$$dCor ( X , Y ) = \frac{dCov ( X , Y )}{\sqrt{dVar ( X ) dVar ( Y )}}$$

While Pearson correlation uses covariance between values as:

$${Cor ( X , Y )}^{2} = \frac{Cov ( X , Y )}{\sqrt{{σ}_{X} {σ}_{Y}}}$$

with covariance calculated as: (X, Y)=Σᵢ₌₁ⁿ(xᵢ−x̅)(yᵢ-y̅); distance correlation depends on the distance covariance determined as:

$${dCov ( X , Y )}^{2} = \frac{1}{{n}_{2}} {\sum }_{j = 1}^{n} {\sum }_{k = 1}^{n} {A}_{j , k} {B}_{j , k}$$

with A and *B as simple linear functions of the pairwise distances between elements in samples X and Y. A and B are doubly centered distance matrices for variables X and Y respectively calculated from the pairwise distance between elements in each sample set with A_{j,k}=a_{j,k}−aⱼ−aₖ+a where a_{j,k}=\sqrt{(xⱼ−xₖ)²} and aⱼ and aₖ respectively the j-row and k-column mean and a the overall mean of A. B includes equivalent measures to A for variable Y. Distance correlation calculation was written in-house under MATLAB using pdist2 to calculate distances between features with Euclidian distance. Correlation was calculated between all features separately for sample groups. Function corr provides the p-value for the correlation measure. For distance correlation we calculated p-value using Student's t cumulative distribution function (tcdf function in MATLAB). Distance correlation values with p>0.01 were set to zero. Sign of distance correlation was equated to the sign of Pearson correlation calculation as proposed by (Pardo-Diaz et al. 2021) and implemented in-house.

### Results

### Lipidomic Profiles of COVID-19 Patients

The lipidomic profile of 21 COVID-19 patients was investigated at several time points from their entry to the hospital followed by sample collection at day 2, 4, 8 and 12. Patients in this cohort experienced different disease trajectories in terms of both disease severity levels and ultimate disease outcome. Age and sex matched control samples involved pre-COVID healthy control samples collected before the COVID-19 pandemic, pre-COVID hospitalized controls including patients with dementias as well as post-COVID healthy control samples collected during the COVID-19 pandemic. Plasma lipidome profiles were measured using the same analytical protocol for all groups. Overview of patient groups used in the study are shown in Table 1. Mortality rate of COVID-19 patients in this group was 28% (6 out of 21 patients).

TABLE 1
Demographic of subject cohorts in the study.

| | | pre-COVID | pre-COVID | post-COVID |
|---|---|---|---|---|
| | COVID-19 | healthy | hospitalized | healthy |
| Characteristics | Cohort | controls | controls | controls |
| Number of | 16/5 | 15/5/1* | 32/10 | 2/3 |
| unique | | | | |
| subjects | | | | |
| (Male/Female) | | | | |
| Number of | 41/9 | 15/5/1* | 32/10 | 2/3 |
| measurements | | | | |
| (Male/Female) | | | | |
| Mean age ± SD | 63.8 ± 16.4 | 64.4 ± 11.7 | 66.4 ± 11.3 | 40.6 ± 13.8 |
| (years) | | | | |

*unidentified

In all samples 90 plasma sphingolipids were measured using HPLC-ESI-MS/MS. Lipid feature assignment and quantification particularly focused on ceramides because of their importance in the immune and inflammatory response (Sokolowska and Blachnio-Zabielska 2019). Lipids were annotated and quantified using previously described methodology (Xu et al. 2013).

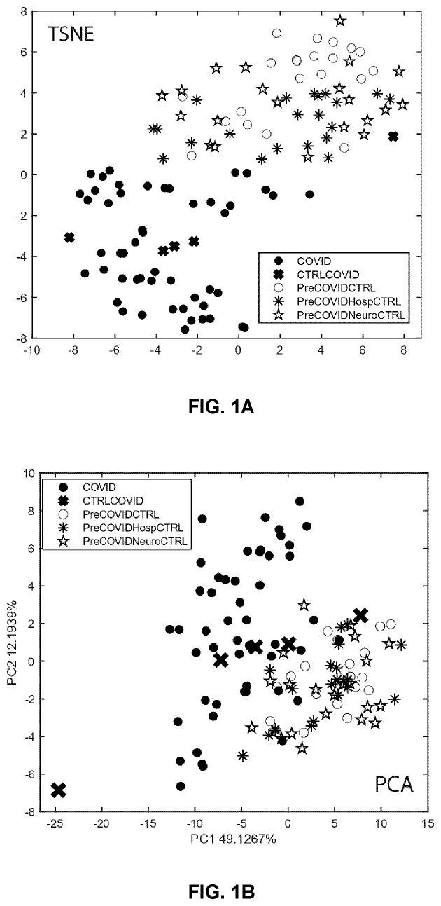

### Stochastic Neighbour Embedding (T-SNE) (FIG. 1A) and Principal Component Analysis (PCA)

Visualization of data using t-SNE (FIG. 1A) and PCA (FIG. 1B) shows that based on major variances lipidomics profiles in COVID-19 subjects are distinct from most samples in other groups. Further selection of significantly different features between COVID-19 patients and all other groups have been determined using an ensemble of methods in order to obtain the best and the smallest set of biomarkers that is independent of the feature selection or analysis methodology including non-correlated features only.

### Feature Selection

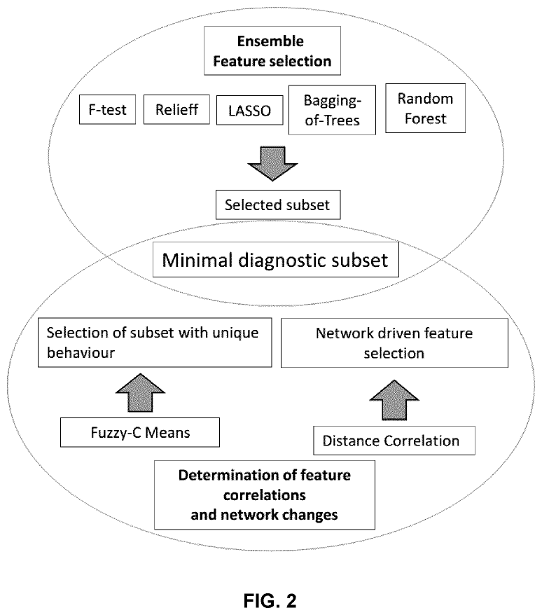

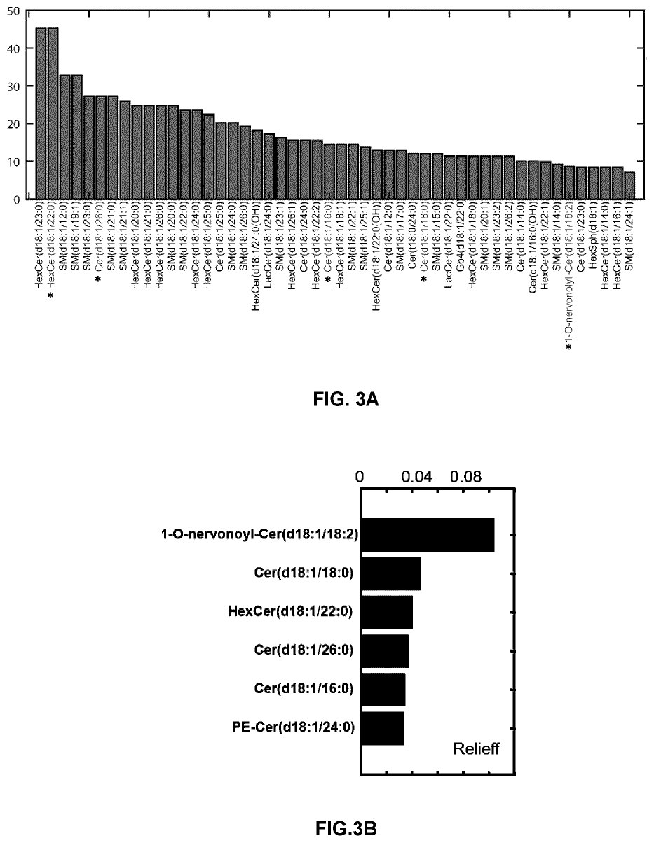

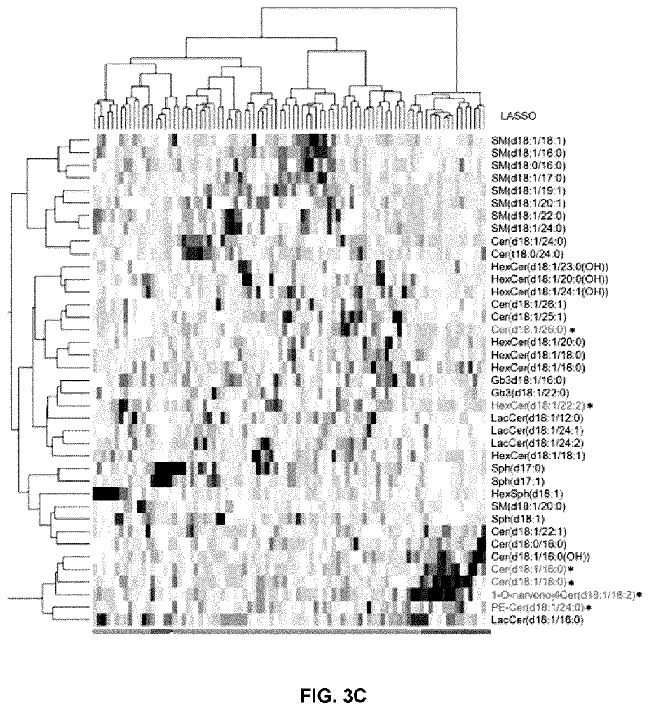

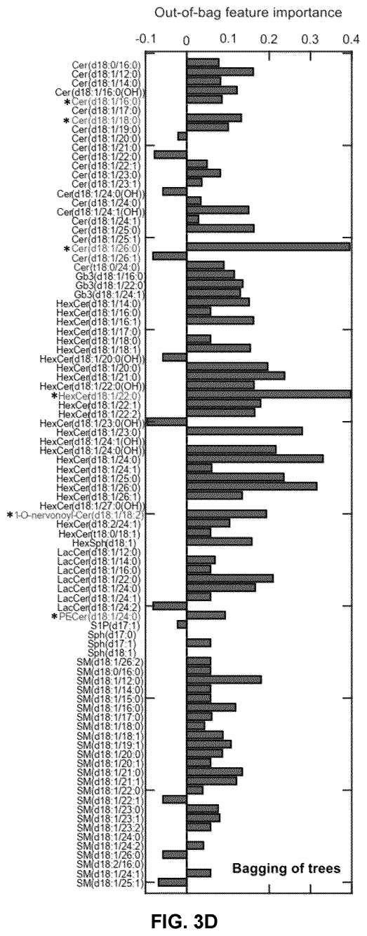

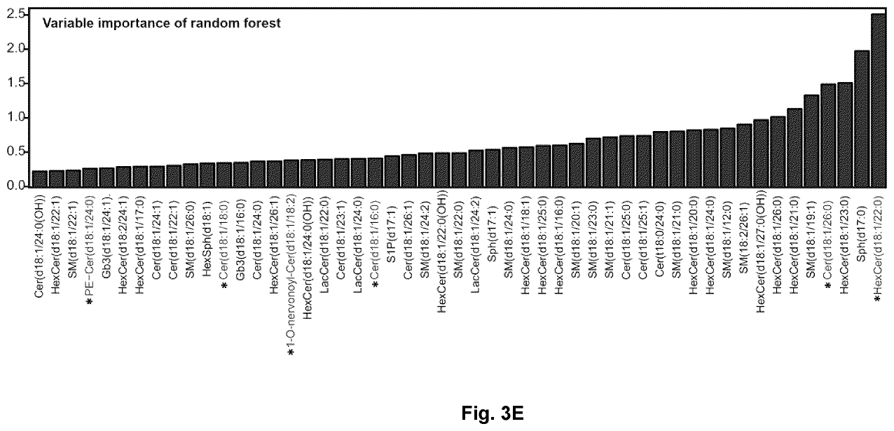

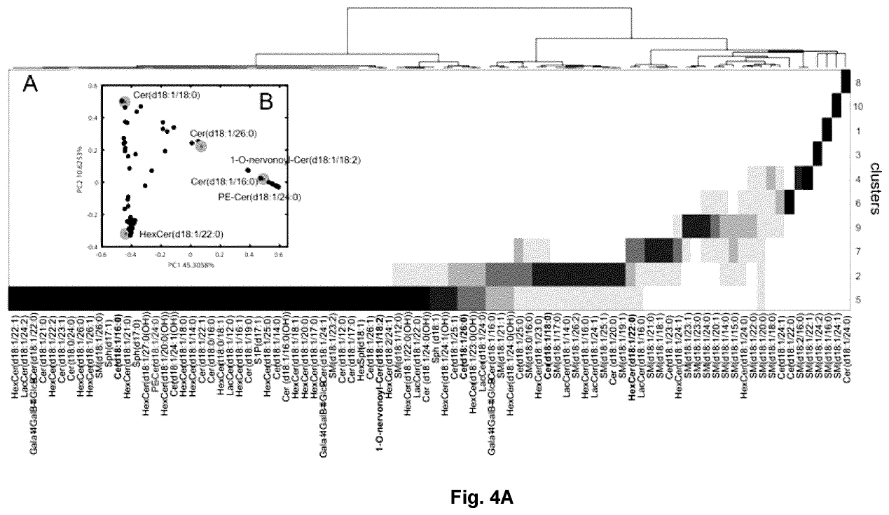

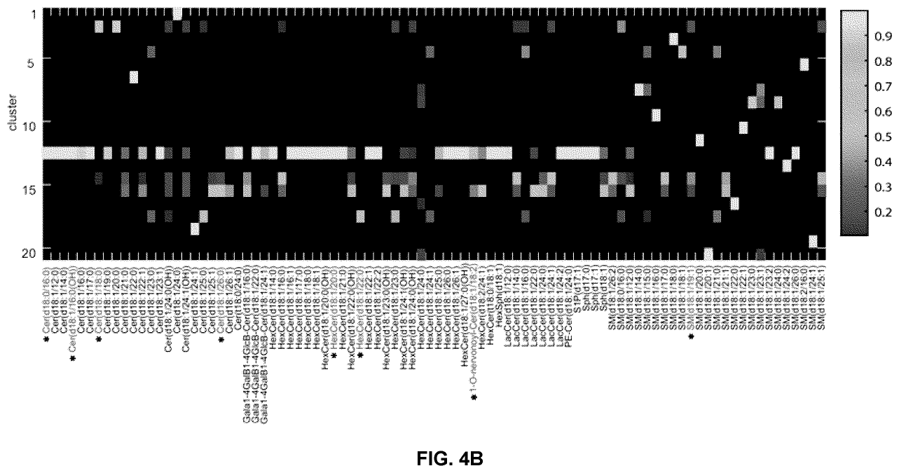

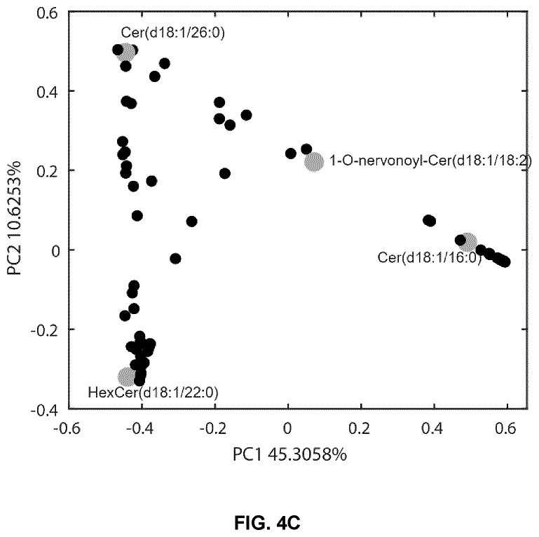

Outline of the feature selection approaches as well as overall methodology used herein for the selection of the minimal biomarker feature set is shown in FIG. 2. Feature selection was performed on z-score normalized data using analysis of statistical significance with F-statistical test (FIG. 3A) as well as machine learning methods Relieff (FIG. 3B), lasso (FIG. 3C), bagging of trees (FIG. 3D) and random forest (FIG. 3E). The ensemble of methods is used to provide selection of the most significant features regardless of the classification methodology of choice. The selection of methodologies used here followed general principles of ensemble selection with approaches including wrapper (lasso), filter (Relieff) and embedded methods (bagging of trees and random forest). All methods analyzed the same dataset using the Heterogeneous ensemble approach (“Ensembles for Feature Selection: A Review and Future Trends” 2019) (https://www.sciencedirect.com/science/article/pii/S1566253518303440). Although there are differences in the top features selected by these methods, a number of features co-occur in different selected groups and are indicated with asterisks in FIG. 4A and FIG. 4C.

### F-Statistics

The first feature significance test utilized here is based on the univariate analysis with F-statistics (FIG. 3A). In this approach each feature is explored separately with the hypothesis that the response values grouped based on the predictor variables are selected from the populations with the same mean against the alternative hypothesis that the population means are not the same. The weight provided by the F-test is equal to −log (p). Selected are features that have weight over 6.9 (equivalent to p<0.001), and thus these 72 out of 90 features have highly statistically significant concentration difference between groups. F-statistics was performed on the first time point collection for COVID-19 cohort and matching control groups.

### Relieff

The Relieff method is more selective, having identified only 6 features having weight >0.02 and selected as significant (FIG. 3B). Relieff is a filter-based feature selection method sensitive to feature interactions. Filter methods use a proxy measurement obtained from the general characteristics of the data to score features (Urbanowicz, 2018). Relieff is a unique filter based algorithm capable of detecting feature dependencies through utilization of nearest neighbors. The Relieff method provides ranking, weight of features, based on their ability to distinguish between k-neighbors for an instance that belong to another class and the ones that are part of the same class group. Relieff analysis was performed on all COVID-19 data, including different sample times for the same patients relative to all non-COVID-19 subjects (FIG. 3B) as well as individual four groups (data not shown) leading to very similar selection of major correlation differences. The lipids selected as significant are: 1-O-nervonoyl-Cer(d18:1/18:2), Cer(d18:1/18:0), HexCer(d18:1/22:0), Cer(d18:1/26:0), Cer(d18:1/16:0) and PE-Cer(d18:1/24:0).

### Lasso

Lasso regression provides selection through removal of features that are not useful or are redundant for classification. In this way lasso presents a different approach to feature selection to the other methods however still all features selected by Relieff are identified as significant by lasso analysis when including only the first time point measurements of COVID-19 subjects (FIG. 3C) and all COVID-19 measurements (data not shown).

### Bagging of Trees and Random Forest

Bagging of trees (FIG. 3D) and random forest (FIG. 3E) methods select features based on their power in decision tree construction with bootstrapping. In the bagging of trees approach, the system creates 300 random sub-samples of the dataset with replacement and then for each set calculates the classification and regression tree model and from all the models calculates the average prediction significance for each feature. For these calculations trees are grown to 5 leaf-node levels and trees are not pruned. This approach uses greedy algorithm to develop trees and thus the decision trees can have structural similarities leading to correlation in prediction. The Random Forest algorithm, by allowing search for sub-trees in only randomly selected sets of features in every random sample set, reduces correlation between predictions. Both methods provide weights, variable importance in the classification, both ranking Hex (d18:1/22:0) highest and with bagging of trees placing Cer(d18:1/26:0) at equally high prediction weight. Both methods rank features selected by Relieff highly with PE-Cer(d18:1/24:0) scoring lowest of the six Relieff features.

Relieff provided the smallest feature set, with all Relieff selected lipids also being present in subsets proposed by other approaches. As a specific combination of minimal sets of features that can distinguish between COVID-19 patients and other groups of subjects at any stage of the disease development as well as any severity level are searched, only features that are selected by distinct methods are included for further consideration.

### Fuzzy C—Means Clustering

Further analysis focused on selecting the most appropriate features from the Relieff group. One criteria was to exclude features that are strongly correlated or show similar behaviour as they do not provide additional information. This further analysis was performed using Fuzzy C-means (FCM) clustering and Distance correlation analysis. Features of particular interest are six lipid molecules determined by all utilized feature selection methods as significant, namely 1-O-nervonoyl-Cer(d18:1/18:2), Cer(d18:1/18:0), HexCer(d18:1/22:0), Cer(d18:1/26:0), Cer(d18:1/16:0) and PE-Cer(d18:1/24:0). Results of FCM clustering of all features across all samples grouped in 10 clusters with fuzziness parameter m=2 is shown in FIG. 4A as Hierarchical cluster of membership values, FIG. 4B as heatmap of all membership values for features clusters in 20 clusters and Principal Component Analysis of all membership values (FIG. 4C).

Based on FCM results, Cer(d18:1/16:0) and PE-Cer(d18:1/24:0) showed very similar behaviour across all samples with the same highest cluster membership as well as co-clustering of all their memberships based on both Hierarchical cluster of membership (FIG. 4A) and Principal component analysis (FIG. 4C) of FCM results. For other features, although largest membership value was the same for some (including 1-O-nervonoyl-Cer(d18:1/18:2) and Cer(d18:1/26:0) together with Cer(d18:1/16:0) and PE-Cer(d18:1/24:0)) when considering weaker memberships to all clusters, these features show distinct behavior.

### Correlation Networks

Distance correlations were calculated between all features for all samples in the control and COVID-19 cohort. Strong correlation, with distance correlation over 0.6 at p<0.01, was observed between Cer(d18:1/18:0) and Cer(d18:1/16:0) as well as PE-Cer(d18:1/24:0) and Cer(d18:1/26:0) or Cer(d18:1/22:0). The correlation networks for Cer(d18:1/16:0), Cer(d18:1/26:0) and PE-Cer(d18:1/24:0) for COVID-19 and control groups are shown in FIG. 5.

### Biomarker Panel

Feature correlation and co-clustering analysis showed that Cer(d18:1/18:0) and PE-Cer(d18:1/24:0) have strong correlations and co-clustering with Cer(d18:1/16:0) and Cer(d18:1/26:0). Additionally, based on random forest analysis, out of the six selected lipids, Cer(d18:1/18:0) and PE-Cer(d18:1/24:0) had the lowest ranks, i.e. classification significance. Therefore including Cer(d18:1/16:0), Cer(d18:1/26:0), 1-O-nervonoyl-Cer(d18:1/18:2) and HexCer(d18:1/22:0) in the biomarker panel may be sufficient due to their strong correlation with Cer(d18:1/18:0) and PE-Cer(d18:1/24:0), and their more significant classification role according to all methods utilized herein.

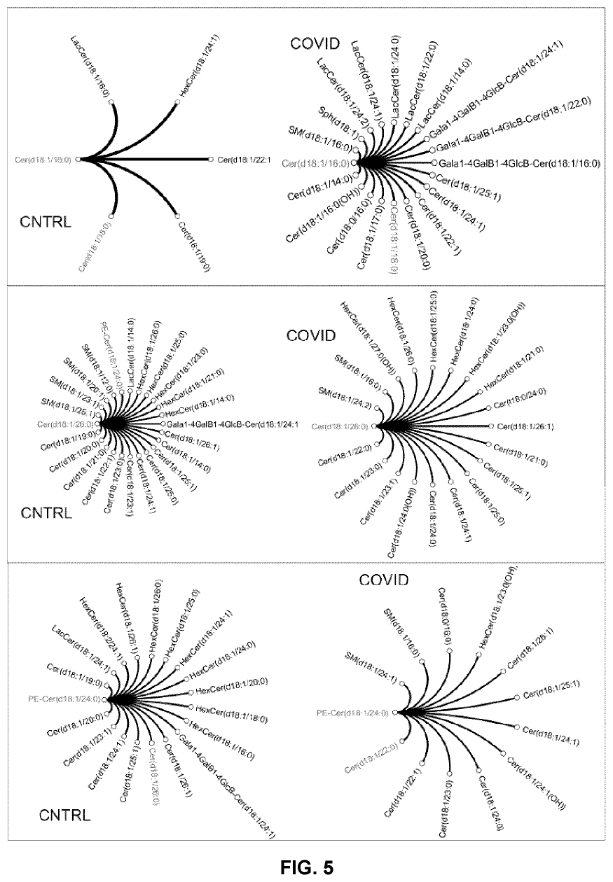

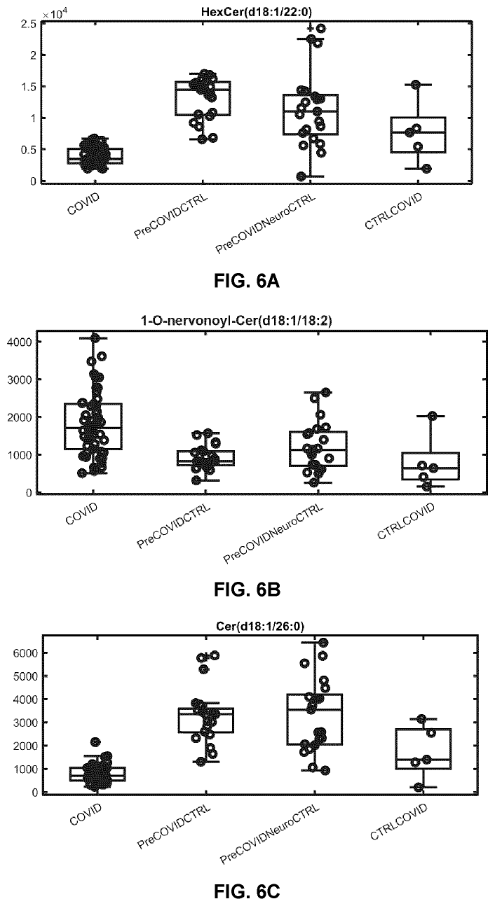

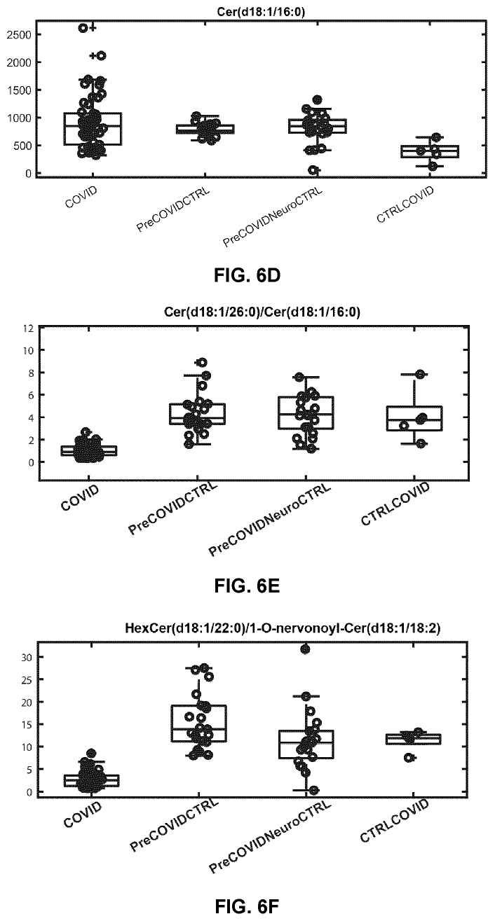

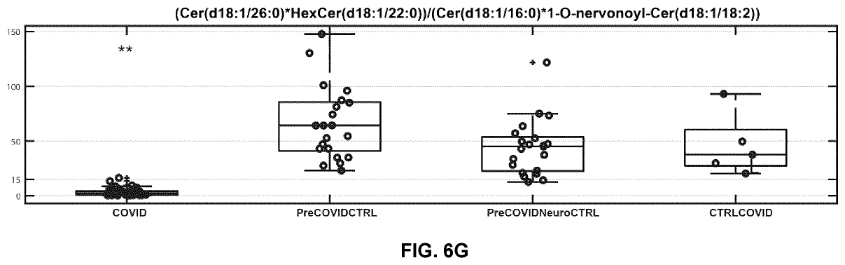

Boxplots showing concentration levels of the four lipids Cer(d18:1/16:0), Cer(d18:1/26:0), 1-O-nervonoyl-Cer(d18:1/18:2) and HexCer(d18:1/22:0), in COVID-19 and control cohorts are shown in FIGS. 6A to D, respectively. As can be seen, the concentration of HexCer(d18:1/22:0) and Cer(d18:1/26:0) was decreased and the concentration of 1-O-nervonoyl-Cer(d18:1/18:2) was slightly increased in the COVID-19 cohort. In order to remove the need for normalization and scaling for these features and to provide a unique diagnostic value that can be clinically utilized, four lipid measurements were used as a ratio across lipid molecule types (ceramides and hexosyl-ceramides) and finally as a product of these ratios. The diagnostic power of the panel is in this way increased leading to highly statistically significant diagnostic power with p value of 2.95*e-25 for differentiation between COVID-19 samples and all the other samples (FIG. 6G).

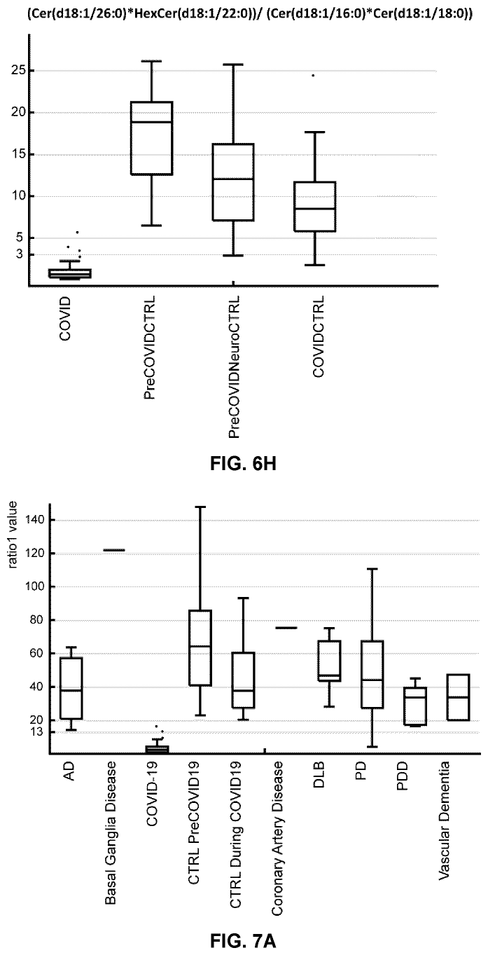

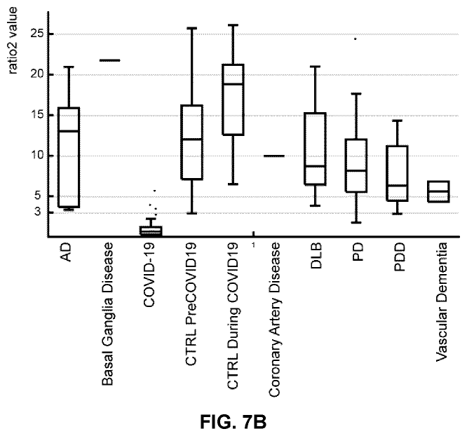

Several requirements were considered in the selection of the biomarker panel. First, individual members of the panel had to be selected as significant features distinguishing between COVID-19 at different disease stages and different groups of non-COVID controls using multiple methods including statistical analysis and machine learning approaches. Second, selected features needed to be largely independent from one another according to fuzzy C-means and correlation analysis. Selected lipids are Cer(d18:1/16:0), Cer(d18:1/26:0), HexCer(d18:1/22:0) and 1-O-nervonoyl-Cer(d18:1/18:2) measured in blood samples. As shown in FIGS. 6E-6H, 7A, and 7B, the following concentration ratios were found to be decreased in COVID-19 samples:
[Cer(d18:1/26:0)]/[Cer(d18:1/16:0)],
[HexCer(d18:1/22:0)]/[1-O-nervonoyl-Cer(d18:1/18:2)],
[Cer(d18:1/26:0)]/[Cer(d18:1/16:0)], and
[HexCer(d18:1/22:0)]/[Cer(d18:1/18:0)].

As can be seen, these concentration ratios are decreased in COVID-19 samples compared to non-COVID-19 a controls. COVID-19 patients had [Cer(d18:1/26:0)]/[Cer(d18:1/16:0)] ratio of 2 or less and non-COVID-19 patients had a [Cer(d18:1/26:0)]/[Cer(d18:1/16:0)] ratio greater than 2. Similarly, COVID-19 patients had a [HexCer(d18:1/22:0)]/[1-O-nervonoyl-Cer(d18:1/18:2)] ratio of 10 or less and non-COVID-19 patients had a [HexCer(d18:1/22:0)]/[1-O-nervonoyl-Cer(d18:1/18:2)] ratio of greater than 10.

Finally, the features were also explored for their power in working in combination that would allow reference free diagnosis of highest accuracy at all disease stages. Concentrations of these lipids for individual plasma samples are combined as:

$$D 1 = \frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$

$$or$$

$$D 2 = \frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$

A diagnostic panel calculated in this way provides accurate classification between patients with COVID-19 at all disease stages relative to all of the other groups including healthy controls samples collected both prior to and during the pandemic as well as hospitalized controls and patients with dementias. According to the dataset presented herein all COVID-19 patients at all stages of the disease have D1<14 and all non-COVID-19 sufferers in all different patient groups have D1>14 providing a cut-off point for the diagnostic set (FIG. 6G). The two samples from the COVID-19 cohort that have the highest D1 values of 16.36 and 13.339 are samples from the same patient with Parkinson's disease in a recovery phase from COVID-19. Also according to the dataset presented herein, all COVID-19 patients at all stages of the disease have D2<3.2 and all non-COVID-19 sufferers in all different patient groups have D2>3.2 providing a cut-off point for the diagnostic set (FIG. 6H). The two samples from the COVID-19 cohort that have the highest D2 values over 3.2 are samples from the same patient with Parkinson's disease in a recovery phase from COVID-19 (as above).

Lipid concentrations were also measured in samples from patients with neurological disorders. Results show that the concentration ratios can be used in COVID-19 classification in patients with neurological disorders including Alzheimer's disease (AD), Dementia Lewy Bodies (DLB), Parkison's disease (PD), Parkinson's disease with dementia (PDD) and Vascular Dementia (FIGS. 7A and 7B).

### CITATIONS FOR REFERENCES REFERRED TO IN THE SPECIFICATION

1. Belacel, N., M. Cuperlovic-Culf, M. Laflamme, and R. Ouellette. 2004. “Fuzzy J-Means and VNS Methods for Clustering Genes from Microarray Data.” Bioinformatics. https://doi.org/10.1093/bioinformatics/bth142.
2. Bezdek, James C. 1981. “Pattern Recognition with Fuzzy Objective Function Algorithms.” https://doi.org/10.1007/978-1-4757-0450-1.
3. Carter, Linda J., Linda V. Garner, Jeffrey W. Smoot, Yingzhu Li, Qiongqiong Zhou, Catherine J. Saveson, Janet M. Sasso, et al. 2020. “Assay Techniques and Test Development for COVID-19 Diagnosis.” ACS Central Science. https://doi.org/10.1021/acscentsci.0c00501.
4. Caterino, Marianna, Monica Gelzo, Stefano Sol, Roberta Fedele, Anna Annunziata, Cecilia Calabrese, Giuseppe Fiorentino, et al. 2021. “Dysregulation of Lipid Metabolism and Pathological Inflammation in Patients with COVID-19.” Scientific Reports 11 (1): 1-10.
5. Danlos, François-Xavier, Claudia Grajeda-Iglesias, Sylvère Durand, Allan Sauvat, Mathilde Roumier, Delphine Cantin, Emeline Colomba, et al. 2021. “Metabolomic Analyses of COVID-19 Patients Unravel Stage-Dependent and Prognostic Biomarkers.” Cell Death & Disease 12 (3): 1-11.
6. Darby, Alistair C., and Julian A. Hiscox. 2021. “Covid-19: Variants and Vaccination.” BMJ 372 (March). https://doi.org/10.1136/bmj.n771.
7. Dunn, J. C. 1973. “A Fuzzy Relative of the ISODATA Process and Its Use in Detecting Compact Well-Separated Clusters.” Journal of Cybernetics. https://doi.org/10.1080/01969727308546046.
8. “Ensembles for Feature Selection: A Review and Future Trends.” 2019. An International Journal on Information Fusion 52 (December): 1-12.
9. Fraser, Douglas D., Marat Slessarev, Claudio M. Martin, Mark Daley, Maitray A. Patel, Michael R. Miller, Eric K. Patterson, et al. 2020. “Metabolomics Profiling of Critically Ill Coronavirus Disease 2019 Patients: Identification of Diagnostic and Prognostic Biomarkers.” Critical Care Explorations 2 (10): e0272.
10. Gostin, Lawrence O. 2021. “9 Steps to End COVID-19 and Prevent the Next Pandemic: Essential Outcomes From the World Health Assembly.” JAMA Health Forum 2 (6): e211852-e211852.
11. Harmon, Stephanie A., Thomas H. Sanford, Sheng Xu, Evrim B. Turkbey, Holger Roth, Ziyue Xu, Dong Yang, et al. 2020. “Artificial Intelligence for the Detection of COVID-19 Pneumonia on Chest CT Using Multinational Datasets.” Nature Communications 11 (1): 1-7.
12. Julkunen, Heli, Anna Cichońska, P. Eline Slagboom, Peter Würtz, and Nightingale Health UK Biobank Initiative. 2021. “Metabolic Biomarker Profiling for Identification of Susceptibility to Severe Pneumonia and COVID-19 in the General Population,” May. https://doi.org/10.7554/eLife.63033.
13. Kononenko, Igor, and Matjaz Kukar. 2007. Machine Learning and Data Mining. Horwood Publishing.
14. Kononenko, I., and E. Simec. 1995. “Induction of Decision Trees Using Relieff.” Proceedings of the ISSEK94 Workshop on Mathematical and Statistical Methods in Artificial Intelligence. https://doi.org/10.1007/978-3-7091-2690-5_14.
15. Liu, Di, Xi Chu, Hao Wang, Jing Dong, Si-Qi Ge, Zhong-Yao Zhao, Hong-Li Peng, et al. 2018. “The Changes of Immunoglobulin G N-Glycosylation in Blood Lipids and Dyslipidemia.” Journal of Translational Medicine 16 (1): 1-10.
16. Majumder, Joydeb, and Tamara Minko. 2021. “Recent Developments on Therapeutic and Diagnostic Approaches for COVID-19.” The AAPS Journal 23 (1): 1-22.
17. Miller, T. E., Garcia Beltran Wf, A. Z. Bard, T. Gogakos, M. N. Anahtar, M. G. Astudillo, D. Yang, et al. 2020. “Clinical Sensitivity and Interpretation of PCR and Serological COVID-19 Diagnostics for Patients Presenting to the Hospital.” FASEB Journal: Official Publication of the Federation of American Societies for Experimental Biology 34 (10). https://doi.org/10.1096/fj.202001700RR.
18. Pardo-Diaz, Javier, Lyuba V. Bozhilova, Mariano Beguerisse-Diaz, Philip S. Poole, Charlotte M. Deane, and Gesine Reinert. 2021. “Robust Gene Coexpression Networks Using Signed Distance Correlation.” Bioinformatics 37 (14): 1982-89.
19. Poss, Annelise M., J. Alan Maschek, James E. Cox, Benedikt J. Hauner, Paul N. Hopkins, Steven C. Hunt, William L. Holland, Scott A. Summers, and Mary C. Playdon. 2020. “Machine Learning Reveals Serum Sphingolipids as Cholesterol-Independent Biomarkers of Coronary Artery Disease.” The Journal of Clinical Investigation 130 (3): 1363-76.
20. “Relief-Based Feature Selection: Introduction and Review.” 2018. Journal of Biomedical Informatics 85 (September): 189-203.
21. Sapkota, Dipak, Tine Merete Søland, Hilde Kanli Galtung, Lars Peter Sand, Simone Giannecchini, Kelvin K. W. To, Maria Cassia Mendes-Correa, Daniel Giglio, Bengt Hasséus, and Paulo Henrique Braz-Silva. 2021. “COVID-19 Salivary Signature: Diagnostic and Research Opportunities.” Journal of Clinical Pathology 74 (6): 344-49.
22. Sokolowska, Emilia, and Agnieszka Blachnio-Zabielska. 2019. “The Role of Ceramides in Insulin Resistance.” Frontiers in Endocrinology 0. https://doi.org/10.3389/fendo.2019.00577.
23. Song, Jin-Wen, Sin Man Lam, Xing Fan, Wen-Jing Cao, Si-Yu Wang, He Tian, Gek Huey Chua, et al. 2020. “Omics-Driven Systems Interrogation of Metabolic Dysregulation in COVID-19 Pathogenesis.” Cell Metabolism. https://doi.org/10.1016/j.cmet.2020.06.016.
24. Székely, Gábor J., Rizzo, Maria N., Bakirov, Nail K. 2007. “Measuring and testing dependence by correlation of distances”. Ann. Statistics. 35 (6): 2769-2794.
25. Theken, Katherine N., and Garret A. FitzGerald. 2021. “Bioactive Lipids in Antiviral Immunity.” Science 371 (6526): 237-38.
26. Tibshirani, Robert. 1996. “Regression Shrinkage and Selection Via the Lasso.” Journal of the Royal Statistical Society: Series B (Methodological). https://doi.org/10.1111/j.2517-6161.1996.tb02080.x.
27. Wang, Wenling, Yanli Xu, Ruqin Gao, Roujian Lu, Kai Han, Guizhen Wu, and Wenjie Tan. 2020. “Detection of SARS-CoV-2 in Different Types of Clinical Specimens.” JAMA: The Journal of the American Medical Association 323 (18): 1843-44.
28. Weissleder, Ralph, Hakho Lee, Jina Ko, and Mikael J. Pittet. 2020. “COVID-19 Diagnostics in Context.” Science Translational Medicine 12 (546). https://doi.org/10.1126/scitransimed.abc1931.
29. Xu, Hongbin, Nicolas Valenzuela, Stephen Fai, Daniel Figeys, and Steffany A. L. Bennett. 2013. “Targeted Lipidomics-Advances in Profiling Lysophosphocholine and Platelet-Activating Factor Second Messengers.” The FEBS Journal 280 (22): 5652-67.
30. Nolen, L. T., Mukerji, S. S. & Mejia, N. I. Post-acute neurological consequences of COVID-19: an unequal burden. Nat Med 28, 20-23 (2022).

What is claimed is:

1. A method of diagnosing, detecting or screening for coronavirus disease 2019 (COVID-19) in a subject, the method comprising:
a) obtaining a blood sample that has been obtained from the subject;
b) measuring concentration of at least two sphingolipids in the blood sample, the sphingolipids being selected from Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0);
c) determining a ratio of the concentration of the at least two sphingolipids;
d) comparing the ratio with a reference value, wherein a differential between the ratio and the reference value is indicative that the subject has COVID-19.

2. The method of claim 1, wherein the concentrations of Cer(d18:1/26:0) and Cer(d18:1/16:0) are determined.

3. The method of claim 2, wherein a [Cer(d18:1/26:0)]/[Cer(d18:1/16:0)] ratio is determined and when the [Cer(d18:1/26:0)]/[Cer(d18:1/16:0)] ratio is less than a reference value of 2, the subject is identified as having or likely having COVID-19.

4. The method of claim 1, wherein the concentrations of HexCer(d18:1/22:0) and 1-O-nervonoyl-Cer(d18:1/18:2) are determined.

5. The method of claim 4, wherein a [HexCer(d18:1/22:0)]/[1-O-nervonoyl-Cer(d18:1/18:2)] ratio is determined and when the [HexCer(d18:1/22:0)]/[1-O-nervonoyl-Cer(d18:1/18:2)] ratio is less than a reference value of 10, the subject is identified as having or likely having COVID-19.

6. The method of claim 1, wherein the concentrations of Cer(d18:1/16:0), Cer(d18:1/26:0), 1-O-nervonoyl-Cer(d18:1/18:2) and HexCer(d18:1/22:0) are determined.

7. The method of claim 6, wherein a
$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$
ratio is determined and when the
$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ 1 - O - nervonoyl - Cer ( d 18 : 1 / 18 : 2 ) ]}$$
ratio is less than a reference value of 14, the subject is identified as having or likely having COVID-19.

8. The method of claim 1, wherein the concentrations of Cer(d18:1/26:0), HexCer(d18:1/22:0), Cer(d18:1/16:0), and Cer(d18:1/18:0) are determined.

9. The method of claim 8, wherein a
$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$
ratio is determined and when the
$$\frac{[ Cer ( d 18 : 1 / 26 : 0 ) ] * [ HexCer ( d 18 : 1 / 22 : 0 ) ]}{[ Cer ( d 18 : 1 / 16 : 0 ) ] * [ Cer ( d 18 : 1 / 18 : 0 ) ]}$$
ratio is less than a reference value of 3.2, the subject is identified as having or likely having COVID-19.

10. The method of claim 1, wherein the concentrations of three sphingolipids are determined, the concentrations of four sphingolipids are determined, the concentrations of five sphingolipids are determined, or the concentrations of six sphingolipids are determined.

11. (canceled)

12. (canceled)

13. (canceled)

14. The method of claim 1, wherein the blood sample is a whole blood sample, a blood serum sample or a blood plasma sample.

15. The method of claim 1, wherein the blood sample is a blood plasma sample.

16. The method of claim 1, wherein the sphingolipid concentrations are measured using mass spectrometry; chromatography optionally liquid chromatography; thin-layer chromatography; gas chromatography; nuclear magnetic resonance spectroscopy; fluorescence spectroscopy; dual polarization interferometry; a high performance separation method; an immunoassay, optionally an enzyme-linked immunosorbent assay; and/or with a binding moiety capable of specifically binding the sphingolipid analyte.

17. The method of claim 1, wherein the sphingolipid concentrations are measured using mass spectrometry.

18. The method of claim 1, wherein the subject has an underlying disease.

19. The method of claim 18, wherein the underlying disease is a neurological disease.

20. The method of claim 19, wherein the neurological disease is Mild Cognitive Impairment (MCI), Parkinson's disease (PD), Parkinson's disease with dementia (PDD), Dementia Lewy Bodies (DLB) disease, or Alzheimer's disease (AD).

21. The method of claim 1, wherein the reference value is derived from a healthy subject or a population of healthy subjects, optionally wherein the subject(s) is/are matched for age, sex and/or underlying disease.

22. The method of claim 1, further comprising administering a treatment suitable for treatment for treating COVID-19 or a complication related thereto to the subject identified as having or likely having COVID-19.

23. A kit comprising internal standards for mass spectrometry analysis for quantification of two or more sphingolipids selected from Cer(d18:1/16:0), Cer(d18:1/26:0), 1-O-nervonoyl-Cer(d18:1/18:2), HexCer(d18:1/22:0), Cer(d18:1/18:0) and PE-Cer(d18:1/24:0), and optionally comprising calibration standards, quality controls, test samples for assay validation and/or instructions for use thereof.
